(19) United States
(12) Patent Application Publication
(10) Pub. No.: US 2006/0008449 A1
(43) Pub. Date: Jan. 12, 2006

(54) Device and methods for treatment of necrotic tissue using stem cells

(76) Inventor: Jason R. Van Tassel, Irvine, CA (US)

Correspondence Address: Donald Bollella;DB Technical Consulting, 126 Almador, Irvine, CA, 92614, US

(21) Appl. No.: 11/149,960

(22) Filed: Jun. 10, 2005

Related U.S. Application Data
(60) Provisional application No. 60/578,510, filed on Jun. 10, 2004.

(51) Int. Cl. A61K 48/00; C12N 5/08

(52) U.S. Cl. 424/93.21; 435/366

(57) ABSTRACT

Devices and methods for precisely directing a dosage of therapeutic substance are disclosed. The devices include an elongated shaft member, a deployable needle associated with the elongated shaft member, an injection reservoir in fluid communication with the deployable needle; and means associated with the deployable needle for displacing the distal end thereof beyond the distal end of the elongated shaft member so that when the elongated shaft member is inserted into a respective area of the human anatomy, the distal end of the deployable needle may be deployed to allow a respective dose of a therapeutic substance to be discharged from the injection reservoir into a desired target area of the anatomy. In one embodiment the therapeutic substance is stem cells applied to damaged tissue. Methods include a medical procedure performed with one particular embodiment of the present invention to inject stem cells into a precise location within the pericardial space of a patient who has experienced myocardial infarction.

### CROSS-REFERENCE TO RELATED APPLICATIONS

The present application claims the benefit of priority of U.S. Provisional Patent Application Ser. No. 60/578,510 filed Jun. 10, 2004 which is incorporated herein by reference in its entirety.

### BACKGROUND OF THE INVENTION

1. Field of the Invention

This invention relates in general to medical devices and methods and, in particular, to stem cell therapies. More specifically, but without restriction to the particular embodiments hereinafter described in accordance with the best mode of practice, this invention relates to devices and methods for the treatment of necrotic tissue using stem cells.

2. Related Background Discussion

Most stem cell research and development has concentrated on “what can stem cells fix?” as opposed to “what is the most efficient way to deliver stem cells?”. This is a reflection of an industry in its infancy. Today, stem cells have primarily been utilized in direct bone marrow transplantation in selective patients with certain types of hematologic malignancies, thus limiting their delivery process to direct graft placement. However, the unlimited potential of stem cells is only beginning to be realized as recent studies have revealed exciting evidence hovering between scientific breakthrough and medical miracle.

Current scientific data supports the fact that stem cells hold the promise for the treatment, cure, and possibly prevention of numerous afflictions, such as heart disease, Parkinson's Disease and spinal cord injuries. Public debate has slowed advancement of the applicability and multi-functionality of stem cells because of the concern over the use of embryonic stem cells. Recent data, however, has indicated that peripheral stem cells when re-introduced to the donor may provide advantages similar to embryonic stem cells without ethical controversy or the risk of rejection. Studies have demonstrated the promise of peripheral blood stem cells for the regeneration of heart tissue and stimulation of angiogenesis (growth of new blood vessels) by injection of stem cells into animals with experimental heart disease. This has also been demonstrated in small clinical trials on humans and, most recently, in Michigan where a youth with traumatic heart damage received an injection of stem cells into his heart. The patient's physician reported the boy's heart efficiency increased over the subsequent weeks. The impact of this technology on the management of heart disease is profound, yet there is currently no system in the marketplace that allows for efficient targeted delivery of stem cells into diseased myocardium. Such a system would increase treatment efficacy by delivering a high concentration of stem cells into the exact location where they are needed.

Coronary heart disease affects 12,900,000 Americans with 1,100,000 new cases each year. In 2000, there were 540,000 heart attacks resulting in 192,898 deaths. The incidence of coronary heart disease, and consequent heart attacks, continues to rise in the United States and is driven by a number of factors. Americans are getting older. Coronary heart disease and heart attacks increase as Americans age due to the accumulation of a lifetime of cardiac insults. Moreover, the 45+ age group accounts for the vast majority of cardiac revascularization procedures. The 2000 U.S. Census estimates that there will be more than 121 million Americans over the age of 45 in 2010, indicating a 24% increase in that age group compared to 2000. Cardiac risk factors continue to proliferate. The increasing incidence of coronary heart disease is essentially guaranteed by the continued high prevalence of cardiovascular disease risk factors, such as obesity, smoking, hypertension, hypercholesterolemia, and diabetes mellitus. Thus the need to address these types of ailments with increase over the coming years.

As biomedical technology improves and Americans become increasingly educated about their treatment options, patients are more and more demanding and seek less invasive procedures. This trend has been reflected in the declining annual number of CABGs, a major invasive procedure, in the last five years. In 2000, the less invasive angioplasty procedure outnumbered CABGs 1,025,000 to 519,000.

Cardiac reperfusion treatments have been limited to simply restoring perfusion to heart tissue. Furthermore, it is often necessary to repeat angioplasty and coronary stent procedures due to the phenomenon of restenosis. That is, in addition to blood vessel narrowing associated with continued coronary vessel insults, the procedures themselves can produce an inflammatory reaction in the blood vessel leading to vessel narrowing. Except for stem cell therapy, no other treatments have been found to replace dead heart tissue.

More than 30 experimental studies have been published in reputable medical and scientific journals demonstrating the cardiac regenerative capabilities of stem cells. Stem cell therapy represents the first real hope of regenerating the heart tissue itself. Moreover, stem cell therapy can also lead to angiogenesis, or the formation of new blood vessels to feed the heart. Combined, these regenerative effects will be a drastic improvement over current treatments of merely preventing further damage.

To date, researchers have been necessarily focused on elucidating the complex science of identifying, isolating, and understanding stem cells. The outcome of this body of research has been a tremendous advance in medical science, giving rise to a whole new field of medical treatment. However with the preponderance of stem research dedicated to basic science, very little work has been done in developing clinical delivery systems for stem cells. Until now, there has been no system available for the delivery of stem cells to cardiac tissue.

### OBJECTS AND SUMMARY OF THE INVENTION

It is, therefore, an object of the present invention to enable the delivery of a high concentration of stem cells to a desired area of human anatomy so that the benefits of stem cell therapy may be achieved in situ.

The present invention relates to methods and apparatus for treating necrotic tissue. More specifically, this invention is directed to methods and apparatus for delivering stem cells into necrotic tissue such as heart tissue involved in a myocardial infarction.

Cardiac Stem Cell Delivery System: According to one aspect of the present invention, the present device and methods allow for targeted delivery of stem cells into damaged myocardium (heart muscle) stimulating tissue regeneration and growth of new blood vessels. The device employs novel technology overcoming the problem of nonspecific cell delivery. The device and related methods can be advantageously employed by cardiologists, cardiothoracic surgeons, and hospitals for use in patients with myocardial infarction (MI) as an adjunct to conventional reperfusion therapy. These devices and methods may be readily adapted to organ systems as well.

Market Need: In view of the lack of comparable prior art devices and methods, the need for the present invention is high. The reasons for this are twofold: 1) the increasingly large number of people currently suffering from coronary heart disease and subsequent myocardial infarction and 2) the rising trend of using minimally-invasive procedures in the treatment of coronary heart disease. The 2003 statistics from the American Heart Association report the prevalence and incidence of coronary heart disease (CHD) to be 12,900,000 and 1,100,000, respectively. Cardiovascular procedures for the treatment of CHD continue to rise, having increased 397% since 1979. The total cost of CHD to society is $129.9 billion each year.

Treatments for myocardial infarction depend on severity and may range from medical management to invasive procedures. In 2000, approximately 1.3 million inpatient cardiac catheterizations and over 500,000 percutaneous transluminal coronary angioplasty (PTCA) procedures were performed.

Device Overview: The present invention is directed to systems for targeted delivery of peripheral blood-derived stem cells to diseased tissue for the purpose of regenerating healthy functional tissue. One embodiment of the present invention consists of a family of disposable sheaths that are placed over a rigid fiber optic endoscope. This system is compatible for use with endoscopes from several manufactures. The present device couples the ability of detection with precision delivery of stem cells to damaged tissues, thereby promoting regeneration.

Accurate Detection: The primary method of detection relies on the visual acuity of the endoscopes to which the present device is attached. Damaged myocardium is easily detected by its alerting pale color when compared to the healthy myocardium. When the damage is not easily detectable by visualization alone, as when the damage tissue is deep within the heart, the use of the imbedded microelectrode stimulator located on the distal tip of the present device overcomes this limitation. In these cases, localization is accomplished by emission of small but electrocardiographically detectable impulses in real time that are correlated with standard twelve lead EKG tracing obtained at the time of the injury. Damaged myocardium causes EKG detectable conduction abnormalities that are easily interpreted. Other modalities that may be incorporated into the present system to improve detection capability may include, but are not limited to, ultrasound, light particle emission, and chemical biosensors.

Precise Delivery: The present device incorporates a deployable needle and a reservoir through which the cells are injected. The needle injector deploys at a specified angle from the axis of the endoscope via a slider system, controlled by operator's thumb and penetrates the myocardium at a predetermined distance from the tip of the endoscope for visual confirmation. In one specific embodiment, once this needle has deployed, another collar locking system locks the needle in place to allow the operator to advance the needle into the myocardium and perform the injection.

Delivery Procedure is Simple, Tested, and Minimally Invasive: The procedure by which the endoscope and overlying sheath is positioned in the pericardial space is simple and minimally invasive. It is designed for use by one operator and can be performed by a qualified cardiologist or cardiovascular surgeon in an outpatient setting in less than one hour.

Orthopedics Applications: The present devices and methods may also be applied advantageously for the purposes of repair of arthritic joints, and acute or chronic injuries to bones, cartilage, ligaments, and tendons. Direct injection of therapeutic substances under direct visualization may achieve significant advantage over existing methods for orthopedic care. For instance, in the treatment of torn meninci of the knee, the cartilage is generally debrided away and the joint splinted for a period of time. An alternative approach would be to inject therapeutic substances and tissue glue into the tear for the purposes of repairing the tear and returning the surface to pre morbid condition without the ablative approach that is the current standard of care.

Gynecologic Applications: Direct injection of the ovaries with therapeutic substances under direct visualization may provide treatment for conditions such as infertility whereby the injected substance may correct underlying metabolic, cellular, or other derangements. It may be possible to render a post menopausal woman fertile with injection of cells and or other therapeutic agents that are missing or present in reduced or excess concentration. In addition, injection into benign or malignant tumors with beneficial therapeutic substances may provide the advantage of treating the condition without ablation of the entire ovary with the potential to affect fertility.

Gastrointestinal Applications: The inventions hereof may be further applied to regenerate diseased tissues such as liver and pancreas with the direct injection of therapeutic substances with out performing highly invasive transplant procedures. Transplant tissue may be injected directly into the affected or other organ to augment the function of that organ thereby prolonging the time course to transplant or averting it all together. The direct injection of tissues, cells, and/or other therapeutic substances with minimally invasive techniques for engraftment of autogenous or exogenous tissue may provide significant advantages over existing treatments.

More specifically, the present invention is directed to a device for delivering a therapeutic substance to a target area within the human anatomy. One specific embodiment of the device includes an elongated shaft member having a distal end and a proximal end; a deployable needle associated with the elongated shaft member, the deployable needle having a distal end and a proximal end; an injection reservoir in fluid communication with the deployable needle; and means associated with the deployable needle for displacing the distal end thereof beyond the distal end of the elongated shaft member so that when the elongated shaft member is inserted into a respective area of the human anatomy, the distal end of the deployable needle may be deployed to allow a respective dose of a therapeutic substance to be discharged from the injection reservoir, through the deployed needle, and into a desired target area of the anatomy. In this embodiment, the therapeutic substance may advantageously include stem cells. In one alternative version of this embodiment of the present invention, the elongated shaft member is hollow and is adapted for use in association with a video endoscope. Either of these embodiments may advantageously include an electrode adapted to the distal end of the elongated shaft member. The electrode has a connection to a power supply and EKG machine so that the distal end of the elongated shaft member is thereby enabled to take an EKG reading of tissue in the target area.

According to another aspect of the present invention, there is provided a system for delivering a therapeutic substance to a target area within the human anatomy. This system includes (1) an elongated tube member having a distal end and a proximal end, (2) a deployable needle associated with the elongated shaft member, the deployable needle having a distal end and a proximal end, (3) an injection reservoir in fluid communication with the deployable needle, (4) a video endoscope having a distal imaging end and a proximal operational end, the video endoscope adapted to receive the elongated tube member over the distal end of the video endoscope so that respective distal ends of the elongated tube member and the video endoscope are in register for co-operative operation, and (5) means associated with the deployable needle for displacing the distal end thereof beyond the distal end of the elongated tube member so that when the elongated tube member is inserted into a respective area of the human anatomy, the distal end of the deployable needle may be deployed to allow a respective dose of a therapeutic substance to be discharged from the injection reservoir, through the deployed needle, and into a desired target area of the anatomy. As with the above embodiments, the therapeutic substance used with this system may advantageously include stem cells. The system may further include an electrode adapted to the distal end of the elongated tube member. This electrode is advantageously provided with a connection to a power supply and EKG machine so that the distal end of the elongated tube member is thereby enabled to take an EKG reading of tissue in the target area.

In accordance with yet another aspect of the present invention there is further provided a method for delivering stem cells to a pericardial target area of a patient. One specific embodiment of this method includes the steps of making a small puncture incision in an upper abdomen area of the patient with a detection needle, the detection needle thereby forming a tract between the upper abdomen area and the pericardial target area; then placing a guide wire into the tract between the upper abdomen area and the pericardial target area; then passing at least one dilator over the guide wire to enlarge the tract; next removing the guide wire and inserting through the at least one dilator a video endoscope including a sheath having a deployable needle capable of stem cell delivery; then visually identifying damaged tissue within the pericardial target area with the video endoscope; next activating the needle to inject stem cells into the damaged tissue; and then removing the video endoscope and the at least one dilator. After the removing step is completed, the further step of closing the small puncture incision with at least one suture may be performed. And also after the removing step is completed, the step of observing the patient for a predetermined number of hours prior to discharge may be performed.

### BRIEF DESCRIPTION OF THE DRAWING

Further objects of the present invention together with additional features contributing thereto and advantages accruing therefrom will be apparent from the following description of the preferred embodiments of the invention which are shown in the accompanying drawing wherein:

FIG. 1A is a perspective view of a sheath-type first embodiment of a therapeutic substance delivery device and system according to the present invention which is adapted for use in conjunction with a video endoscope;

FIG. 1B is a perspective view of a second embodiment of a therapeutic substance delivery device which is adapted for stand alone use according to the present invention;

FIG. 2 is an enlarged perspective view of the sheath type device illustrated in FIG. 1A;

FIG. 3A is a perspective schematic view of a human torso showing a first step of a medical procedure performed with the devices of the present invention;

FIG. 3B is another perspective schematic view of a human torso showing a second step of a medical procedure performed with the devices of the present invention;

FIG. 3C is a perspective schematic view similar to FIGS. 3A and 3B showing a third step of a medical procedure performed with the devices of this invention; and

FIG. 3D is a perspective pictorial schematic view with a cut-away section of a human torso representing various steps of a medical procedure performed with one particular embodiment of the present invention to inject stem cells into a precise location within the pericardial space of a patient who has experienced myocardial infarction.

### DETAILED DESCRIPTION OF THE PREFERRED EMBODIMENTS

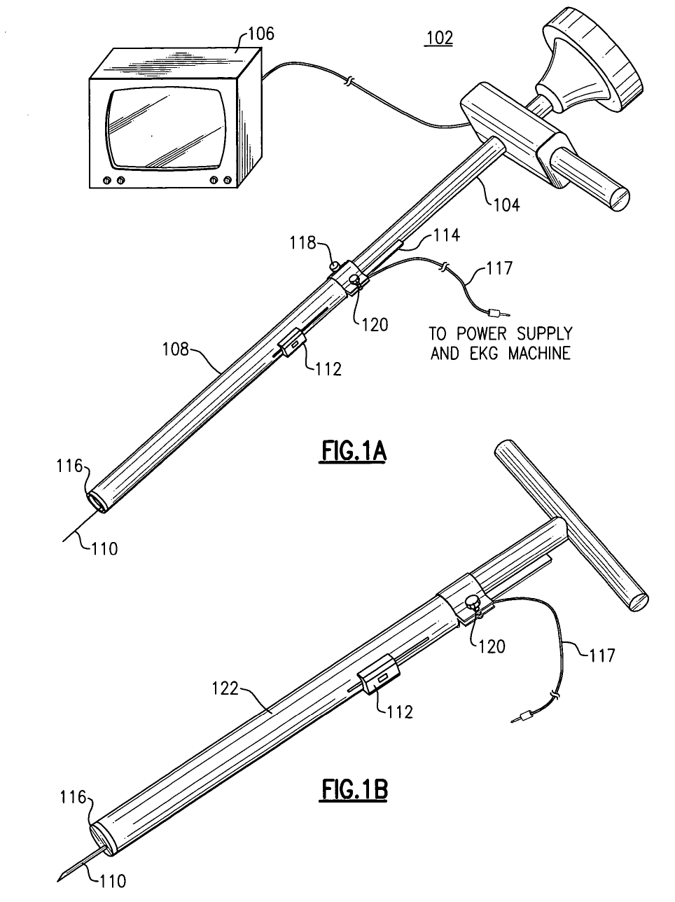

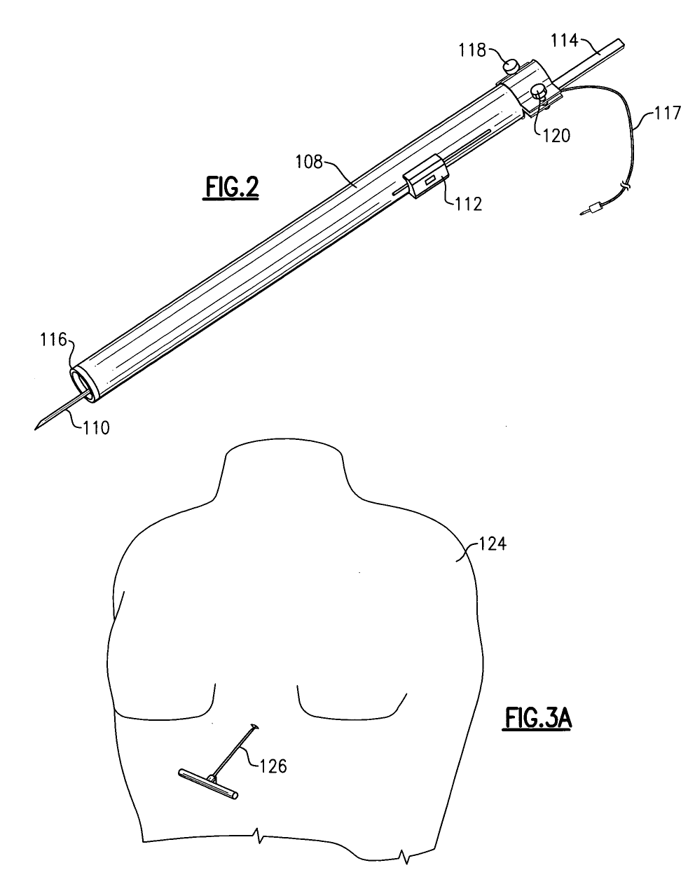

Referring now to FIGS. 1A and 2, there is shown a diagnostic and treatment system 102 according to the present invention. The system 102 includes an endoscope 104 a video monitor 106 and a therapeutic substance dispensing sheath 108. This sheath 108 is provided with a deployable needle 110 and a deployable needle switch 112. As illustrated in FIGS. 1A and 2, the deployable needle 110 includes an injection reservoir 114. In the embodiment of the sheath 108 shown in FIGS. 1A and 2 the distal end of the sheath 108 is provided with an electrode or microelectrode 116. The microelectrode 116 illustrated in this embodiment is annular shape and is in register with the open end of the sheath 108. The electrode 116 is wired to a connector 117 which, as illustrated, is a wire connector that may be plugged into an EKG machine so that the electrode 116 is enabled to take an electrocardiographic reading of human tissue. In an alternate embodiment of the electrode assembly, the electrode 116 may be wired to a wireless transponder/receiver device with an internal power supply so that the connection to the EKG machine may be made wirelessly.

In the embodiment of the sheath 108 shown in FIGS. 1A and 2, the proximal end of the sheath 108 includes a first thumb screw 118 and a second thumb screw 120. As illustrated for exemplary purposes, the sheath 108 is adapted to slide onto the distal end of the endoscope 104 so that the distal end of the endoscope is in register with the distal end of the sheath 108 upon use of the device. The deployable needle 110 as illustrated in FIGS. 1A and 2 is shown in its fully deployed or extended condition. In active use of the device, the switch 112 may be advantageously employed to deploy and retract the needle 110. Upon initial use of the device, the needle 110 would be in the fully retracted position thus not extending beyond the distal end of the sheath or endoscope. Upon use of the device and proper location of the distal ends of both the sheath 108 and the endoscope 104, the operator of the device may activate switch 112 so that the needle 110 is deployed in its fully extended condition as shown in FIGS. 1A and 2. The first thumb screw 118 may be use to adjust the sheath 108 along the length of the endoscope probe 104. In use of the device, a preferred position for distal end of the sheath 108 is in register with the distal end of the endoscope 104. Once this position is obtained the user of the device may then tighten the first thumb screw 118 so that the sheath 108 is locked in a fix manner to the shaft of the endoscope 104. Prior to use of the device, the injection reservoir 114 is loaded with a therapeutic substance which would preferably be liquid in nature and for certain applications would preferably include stem cells. As illustrated in FIGS. 1A and 2, the sheath 108 includes the second thumb screw 120 which may be used as the needle position lock to lock the needle 110 in either the fully deployed position, the fully retracted position, or a partially deployed position.

The system associated device shown in FIGS. 1A and 2 is thus intended to be used for medical purposes in performing diagnostic and therapeutic treatment on animal tissue including human tissue within the animal or human anatomy. The art of using various surgical instruments including trocars, endoscopes, and various other probes, devices, and instruments used in, for example, laparoscopic surgery is well known and advances relating thereto have been contributing to the art of medical surgery. Recently with the advent of the potential therapeutic uses of stem cells, however, such devices have not been adapted for readily applying amounts of stem cells to target tissue areas within the animal or human anatomy. Such medical devices have not kept pace technically with advances in medical research due to the very recent discoveries and appreciation of stem cell therapy. Thus the present system and devices provide the user of endoscopic or laparoscopic surgical equipment and related tools the further advantage of rapidly identifying within the human anatomy target tissue which may be the subject of therapeutic treatment by the application of a wide variety of therapeutic substances which in particular may include stem cells. Thus one aspect of the present invention is the use of the device the reservoir 114 to advantageously include a dose of stem cells. The device is then used to discharge a predetermined dosage of therapeutic stem cells to a location within the animal or human anatomy which had been determined by the medical practitioner through visualization on the monitor 106 by use of the endoscope 104.

With reference now to FIG. 1B there is shown an alternate embodiment to the device according to the present invention. This device illustrated in FIG. 1B includes and elongated shaft member 122 which is intended for stand alone use rather than use in intimate cooperative combination and association with an endoscope such as is the case in the embodiment discussed above in conjunction with FIGS. 1A and 2. With continued reference now to the embodiment illustrated in FIG. 1B the device therein includes the deployable needle 110, the deployable needle switch 112, the microelectrode 116 connected to the wire connector 117 which in turn may be connected to a power supply and EKG machine. The device illustrated in FIG. 1B is used in a similar manner as discussed regarding the device and system illustrated in FIGS. 1A and 2. Thus the device of FIG. 1B may be used either independently or in combination with a separate endoscope or minimally invasive laparoscopic surgical tools. Either of the devices illustrated in FIGS. 1A, 1B, and 2 may include the electrode 116 and connector 117 in alternative embodiments. When the electrode 116 and connector 117 are included as part of the device, the connector 117 may be connected to an EKG machine so that the tissue may be tested and thus diagnosed as either healthy tissue or otherwise damaged tissue due to an abnormal electrocardiographic reading.

According to one of the various methods of the present invention, there is provided a procedure by which the endoscope 104 and overlying sheath 108 is positioned in the pericardial space of a patient. This procedure is simple and minimally invasive. It is designed for use by one operator and can be performed by a qualified cardiologist or cardiovascular surgeon in an outpatient setting in less than one hour.

The procedure consists of a small puncture in the upper abdomen directed toward the patient's heart with an included detection needle. This needle is used to place a guide wire into the pericardial space. A series of dilators are then passed over the guide wire to enlarge the tract. The guide wire is removed and the device is inserted through the largest of the dilators and the surface of the heart is visualized using the video endoscope. The damaged areas are identified visually and/or electrocardiographically, the needle is deployed, and the stem cells are precisely injected. The scope and dilator are then removed and the small incision is closed with suture. The patient is then observed for several hours prior to discharge. The aforementioned procedure is similar to current standard procedures for the treatment of pericardial effusions.

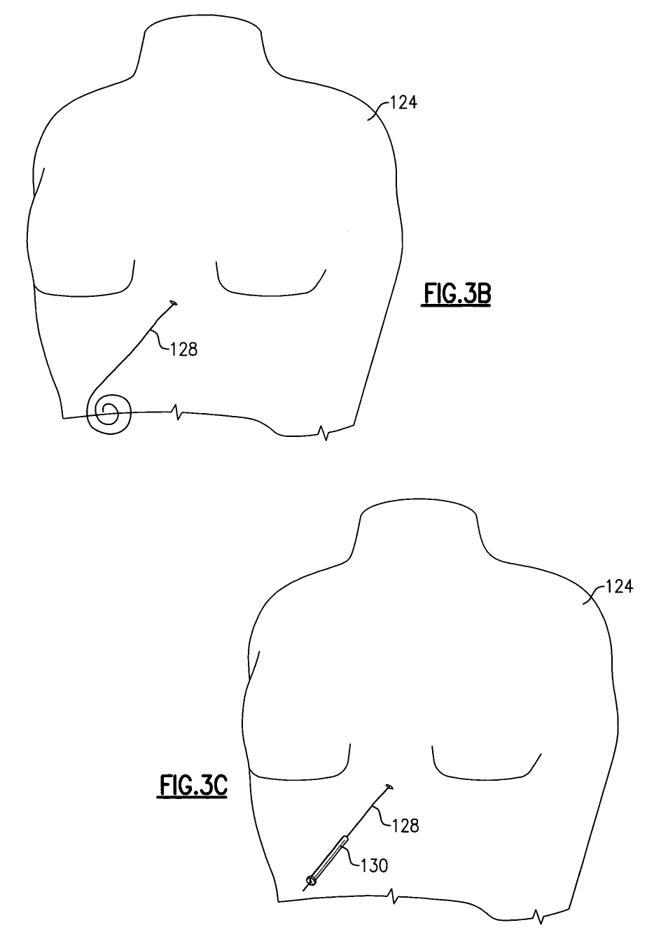

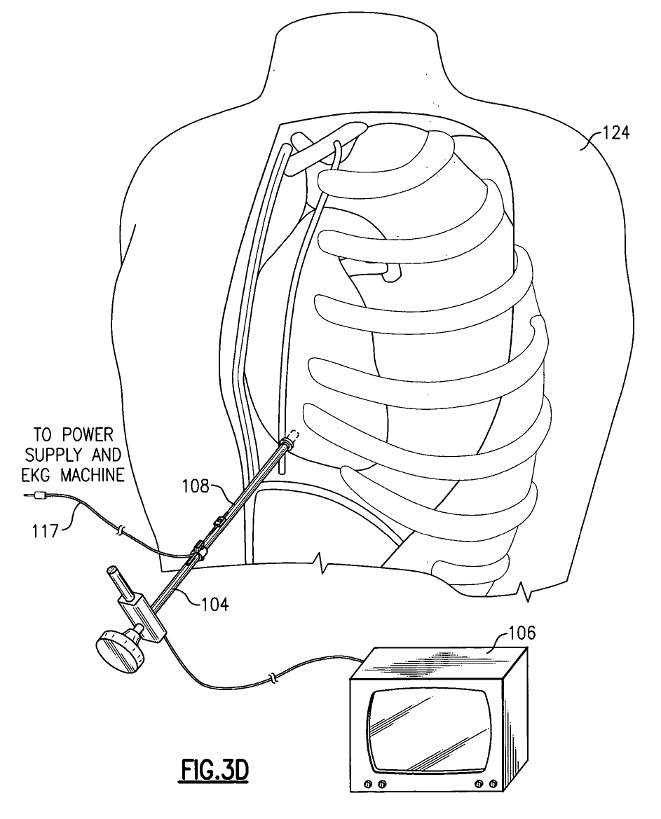

With reference now more specifically to FIGS. 3A-3D there is shown various perspective pictorial views of a human torso and schematic representations of various steps of a medical procedure performed with one particular embodiment of the present invention to inject stem cells into a precise location within the pericardial space of a patient who has experienced myocardial infarction. Firstly, FIG. 3A shows a human torso 124 wherein a small puncture in the upper abdomen has been made with a detection needle 126. Next in FIG. 3B, there is shown a guide wire 128 being inserted into the puncture wound. By the skilled hands of a qualified cardiologist or cardiovascular surgeon, the guide wire 128 is directed toward the pericardial space of the patient. In FIG. 3C a dilator 130 is being positioned over the guide wire 128 to open or enlarge the tract between the external surface of the abdomen and the internal anatomy of the patient. As discussed above, various increasing diameters of dilators 130 may be used to enlarge the tract to a desired size. FIG. 3D further illustrates the torso 124 as shown in cut-away where the first, sheath-type embodiment of the present invention, as illustrated in FIGS. 1A and 2, is employed during surgical use as intended. The damaged areas of the heart tissue are identified visually by use of the endoscope 104 and monitor 106 as shown. Damaged heart tissue may also be diagnosed electrocardiographically by use of the device electrode 116 which is connected to an EKG machine by the connector 117. After the damaged tissue is located, the needle 110 is deployed, and the stem cells are precisely injected.

It should be appreciated by one of skill in the art that all of the compositions and methods disclosed herein can be made and executed without undue experimentation in light of the present disclosure. While this invention has been described in detail with reference to a certain preferred embodiments, it should be understood that the present invention is not limited to those precise embodiments. Rather, in view of the present disclosure which describes the current best mode for practicing the invention, many modifications and variations would present themselves to those of skill in the art without departing from the scope and spirit of this invention. Furthermore, those skilled in the art will recognize, or be able to ascertain, using no more than routine experimentation, many equivalents to the specific embodiments of the invention described herein.

The scope of the invention is, therefore, indicated by the following claims rather than by the foregoing description and all changes, modifications, and variations coming within the meaning and range of equivalency of the claims are to be considered within their scope.

What is claimed is:

1. A device for delivering a therapeutic substance to a target area within the human anatomy, said device comprising:
an elongated shaft member having a distal end and a proximal end;
a deployable needle associated with said elongated shaft member, said deployable needle having a distal end and a proximal end;
an injection reservoir in fluid communication with said deployable needle; and
means associated with said deployable needle for displacing said distal end thereof beyond said distal end of said elongated shaft member so that when said elongated shaft member is inserted into a respective area of the human anatomy, said distal end of said deployable needle may be deployed to allow a respective dose of a therapeutic substance to be discharged from said injection reservoir, through said deployed needle, and into a desired target area of the anatomy.

2. The device according to claim 1 wherein said therapeutic substance includes stem cells.

3. The device according to claim 1 wherein said elongated shaft member is hollow and is adapted for use in association with a video endoscope.

4. The device according to claim 3 wherein said therapeutic substance includes stem cells.

5. The device according to claim 1 further including an electrode adapted to the distal end of said elongated shaft member, said electrode having a connection to a power supply and EKG machine so that said distal end of said elongated shaft member is thereby enabled to take an EKG reading of tissue in the target area.

6. The device according to claim 3 further including an electrode adapted to the distal end of said hollow elongated shaft member, said electrode having a connection to a power supply and EKG machine so that said distal end of said hollow elongated shaft member is thereby enabled to take an EKG reading of tissue in the target area.

7. A system for delivering a therapeutic substance to a target area within the human anatomy, said system comprising:
an elongated tube member having a distal end and a proximal end;
a deployable needle associated with said elongated shaft member, said deployable needle having a distal end and a proximal end;
an injection reservoir in fluid communication with said deployable needle;
a video endoscope having a distal imaging end and a proximal operational end, said video endoscope adapted to receive said elongated tube member over said distal end of said video endoscope so that respective distal ends of said elongated tube member and said video endoscope are in register for co-operative operation; and
means associated with said deployable needle for displacing said distal end thereof beyond said distal end of said elongated tube member so that when said elongated tube member is inserted into a respective area of the human anatomy, said distal end of said deployable needle may be deployed to allow a respective dose of a therapeutic substance to be discharged from said injection reservoir, through said deployed needle, and into a desired target area of the anatomy.

8. The system according to claim 7 wherein said therapeutic substance includes stem cells.

9. The system according to claim 7 further including an electrode adapted to the distal end of said elongated tube member, said electrode having a connection to a power supply and EKG machine so that said distal end of said elongated tube member is thereby enabled to take an EKG reading of tissue in the target area.

10. A method for delivering stem cells to a pericardial target area of a patient, said method comprising the steps of:
making a small puncture incision in an upper abdomen area of the patient with a detection needle, said detection needle thereby forming a tract between said upper abdomen area and said pericardial target area;
placing a guide wire into said tract between said upper abdomen area and said pericardial target area;
passing at least one dilator over said guide wire to enlarge said tract;
removing said guide wire and inserting through said at least one dilator a video endoscope including a sheath having a deployable needle capable of stem cell delivery;
visually identifying damaged tissue within said pericardial target area with said video endoscope;
activating said needle to inject stem cells into said damaged tissue; and
removing said video endoscope and said at least one dilator.

11. The method according to claim 10 wherein after said removing step is completed, further performing the step of closing said small puncture incision with at least one suture.

12. The method according to claim 10 wherein after said removing step is completed, further performing the step of observing the patient for a predetermined number of hours prior to discharge.
